(19) United States
(12) Patent Application Publication
(10) Pub. No.: US 2004/0029188 A1
(43) Pub. Date: Feb. 12, 2004

(54) Method for the detection of human hematopoietic short term repopulating cells

(76) Inventors: Connie J Eaves, British Columbia (CA); Hanno Glimm, Freiburg (DE)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/416,147

(22) PCT Filed: Nov. 7, 2001
(86) PCT No.: PCT/CA01/01555

(51) Int. Cl. G01N 33/53; G01N 33/567

(52) U.S. Cl. 435/7.2; 800/3

(57) ABSTRACT

Two novel populations of human short term repopulating cells are described. In particular, The inventors have shown that sublethally irradiated NOD/SCID-b2M−/− mice allow the efficient engraftment of two previously undescribed populations of human short term repopulating cells (STRC) that do not produce detectable progeny in the more widely used NOD/SCID mouse. These novel cells are designated short term repopulating cells—myeloid (STRC-M) and short term repopulating cells—lympho-myeloid (STRC-ML) to reflect their different lineage potentials. The invention includes an assay for detecting STRC-M and STRC-ML which is useful in a wide range of applications including assessing of the engraftment potential of human hematopoietic cells, testing the toxicity of drugs on hematopoietic cells and in assessing the viability of hematopoietic cells that have been stored and processed.

### FIELD OF THE INVENTION

This invention relates to a novel method for the detection of human hematopoietic short term repopulating cells.

### BACKGROUND OF THE INVENTION

Blood cells are generated throughout adult life from a tiny subpopulation of undifferentiated stem cells. In adults, these stem cells are concentrated in the bone marrow (BM), although at birth they are also present in the blood in relatively high numbers (1-4). Because hematopoietic stem cells can enter the BM from the circulation at high efficiency, (5,6) the intravenous transplants of adult BM cells and more recently, of cord blood (CB) and mobilized blood (mPB) cell harvests, has become an important therapeutic modality for patients with a broad spectrum of malignant and genetic disorders. Nevertheless, in many instances undesirable patterns of hematologic recovery are obtained, including transplants of autologous sources (7). In addition, experiments in model systems indicate a need for improved understanding of the various types of human hematopoietic cells that make up the total transplantable compartment and how changes in their numbers in a given inoculum will affect the kinetics and durability of engraftment to be obtained with transplants that have been previously manipulated ex vivo to expand, purge or genetically modify the cells originally present.

Previous studies in mice have distinguished hematopoietic progenitors with different engraftment properties. Cells with long term reconstituting ability are invariably able to regenerate all hematopoietic lineages and generate progeny capable of repopulating secondary and tertiary recipients (8-10). Other cells with similar differentiation potentialities may reconstitute both lymphoid and myeloid compartments but typically for less than 4 months (9,11). Additional subpopulations of murine cells with myeloid- or lymphoid-restricted reconstituting abilities have been described (12,13).

Evidence of an analogous hierarchy of human hematopoietic cells has been obtained both from in vitro studies (14) and from analyses of human cells transplanted into human sheep in utero (15). However, both of these approaches are limited and neither has proven to be clinically useful. The ability of human hematopoietic cells to engraft the BM of sublethally irradiated NOD/SCID mice with both myeloid and lymphoid progeny within 6 weeks (16,17) and at high efficiency (5,6) has made this model a popular alternative for assessment and characterization of human hematopoietic stem cell phenotypes (3,18). Limiting dilution analyses using this model have shown that the human cell engraftment is quantitative, (3,19) independent of exogenous cytokine administration if sufficient cells are co-injected, (16,17,20) and attributable almost exclusively to the CD38⁻ subset of CD34⁺ cells with unrestricted lympho-myeloid differentiation potential, (3,19) although CD34⁻ human hematopoietic stem cells have also been reported (21,22).

Intravenous transplants of adult bone marrow cells, mobilized peripheral blood and cord blood have become an important therapy for patients with a broad spectrum of malignant and genetic disorders. The transplant graft replaces the patients hematopoiesis which has been compromised due to an existing condition or chemo/radiation therapy. A successful hematopoietic transplant requires both rapid short-term and long-term (life time) maintenance of the entire hematopoietic compartment. Different populations of cells in the hematopoietic graft are responsible for short and long-term repopulation. As both these populations are essential for a successful transplant there is a need for in vivo assays which distinguish between short and long term repopulating potential. Engraftment of human cells in the bone marrow of sublethally irradiated NOD/SCID mice has been used as an in vivo indication of long-term repopulating cells.

There is a need in the art for an in vivo assay which measures the short-term repopulating ability of human cells. Such an assay will enable evaluation of factors affecting patterns of hematologic recovery and characterization of the engraftment potential of clinical transplants in a variety of settings.

### SUMMARY OF THE INVENTION

The present inventors have developed a method that allows the selective detection of previously unrecognized populations of short term repopulating human cells including one with early transient myeloid-restricted potential and another with short-lived lympho-myeloid repopulating activity. The method involves transplanting human hematopoietic cells into nonobese diabetic-severe combined immunodeficiency −β₂ microglobulin null (NOD/SCID-β₂M^{-/-}) mice which allows the efficient engraftment of two previously undescribed populations of human short term repopulating cells (STRC) that do not produce detectable progeny in the more widely used nonobese diabetic-severe combined immunodeficiency (NOD/SCID) mouse. Therefore the invention provides an assay which enables the detection of short term repopulating cells and that provides rapid (3 weeks post transplant) human cell engraftment in the bone marrow of NOD/SCID-β₂M^{-/-} mice.

The present invention includes a method of detecting a short term repopulating human cell that can produce myeloid cells in NOD/SCID-β₂M^{-/-} mice comprising (a) transplanting human hematopoietic cells in a NOD/SCID-β₂M^{-/-} mouse and (b) detecting human erythroid cells at approximately three weeks post-transplant.

The present invention also provides a short term repopulating human cell that can produce myeloid cells in NOD/SCID-β₂M^{-/-} mice. These cells are termed STRC-M herein.

The present invention also includes a method of detecting a short term repopulating human cell that can produce myeloid and lymphoid cells in NOD/SCID-β₂M^{-/-} mice comprising (a) transplanting human hematopoietic cells in a NOD/SCID-β₂M^{-/-} mouse and (b) detecting human myeloid and lymphoid cells at approximately six to eight weeks post-transplant.

The present invention further provides a short term repopulating human cell that can produce lymphoid and myeloid cells in NOD/SCID-β₂M^{-/-} mice. These cells are termed STRC-ML herein.

The invention includes all uses of the methods for detecting the STRC-M and STRC-ML including the use in assessing the engraftment potential of human hematopoietic cells, testing the toxicity of drugs on hematopoietic cells and in assessing the viability of hematopoietic cells that have been stored and processed.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1A is a graph showing total human CD45/71⁺ in a NOD/SCIDβ₂M^{-/-} mice and NOD/SCID mice.

FIG. 1B are bar graphs showing the production of particular hematopoietic lineages as a portion of the total human CD45/71⁺ cells after different periods in a NOD/SCID-β₂M^{-/-} mice and NOD/SCID mice.

FIG. 1C is a representative FACS profile of cells harvested from the bone marrow of a NOD/SCID-β₂M^{-/-} mouse three weeks after transplantation of human Lin⁻ BM cells.

FIG. 2 shows graphs demonstrating the ability of CD34⁺CD38⁺ cells and CD34⁺CD38⁻ cells to engraft a NOD/SCID-β₂M^{-/-} mice.

FIG. 3 are graphs showing that G₀/G₁ and S/G₂/M cells from 5 day expansion cultures of human CB cells show equivalent distributions of repopulating activity in a NOD/SCID-β₂M^{-/-} mice (A) and progenitor numbers detected in vitro (B).

FIG. 4 is a schematic showing a proposed model indicating a hierarchy of transplantable human hematopoietic cells with distinct biological properties.

### DETAILED DESCRIPTION OF THE INVENTION

The inventors have used a strain of immunodeficient mice in which the residual low NK activity present in the NOD/SCID mouse was essentially eliminated by backcrossing the β₂ microglobulin null (β₂M^{-/-}) genotype onto the NOD/SCID background (23). These mice are available from the Jackson Laboratory in Bar Harbor Maine (strain name: NOD-PrKdc^{scid} B2m^{tmlUnc}/J; stock number 002570). Initial studies showed that higher levels of human lympho-myeloid engraftment could be consistently obtained 6-8 weeks post-transplant in these recipients when human cells were injected by comparison to results obtained in NOD/SCID hosts (24). However, as detailed below, the inventors have now determined that NOD/SCID and NOD/SCID-β₂M^{-/-} mice are, in fact, repopulated by different types of human hematopoietic cells. NOD/SCID mice appear to be more selective for more primitive stem cell populations, whereas NOD/SCID-β₂M^{-/-} mice are additionally engrafted by two types of human cells with short term repopulating activity.

Xenotransplantation systems suitable for analyzing the normal and abnormal human transplantable hematopoietic compartment are of pivotal importance for clinical as well as experimental applications. The inventors have shown that sublethally irradiated NOD/SCID-β₂M^{-/-} mice allow the efficient engraftment of two previously undescribed populations of human short term repopulating cells (STRC) that do not produce detectable progeny in the more widely used NOD/SCID mouse. These novel cells are designated short term repopulating cells—myeloid (STRC-M) and short term repopulating cells—lympho-myeloid (STRC-ML) to reflect their different lineage potentials. It is important to note that because of the reduced terminal differentiation and poor peripheralization of human hematopoietic cells that repopulate the BM of either of these mouse strains, the characterization of human xenotransplants in these hosts requires analyses of maturing human cells as they are produced within the BM.

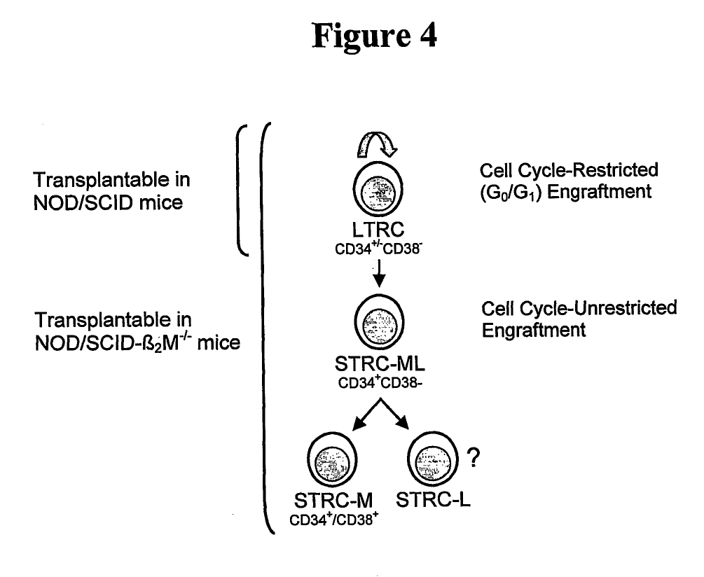

FIG. 4 shows a model of the proposed hierarchy of human repopulating cells that engraft NOD/SCID-β₂M^{-/-} mice. Time course studies of a large series of NOD/SCID-β₂M^{-/-} mice transplanted with multiple sources of human hematopoietic cells, including both fresh and cultured cells, provided the first indication that these mice support a broader range of transplantable human cells than those that reconstitute the closely related NOD/SCID mouse. Long term repopulating cells (LTRC) include CD34⁻CD38⁻ cells (21,36) as well as cells expressing CD34 but not CD38 (3,19). The engraftment ability of LTRC is restricted to the G₀/G₁ phases of the cell cycle (29) and are the only human cells that engraft NOD/SCID mice. STRC-ML are CD34⁺CD38⁻ and their ability to engraft NOD/SCID-β₂M^{-/-} mice is not cell cycle-restricted. Most freshly isolated STRC-M express both CD34 and CD38. The STRC-M and STRC-ML cells are further described below.

### I. Assay for the Detection of Short Term Repopulating Cells

The finding by the present inventors that two previously unknown short term repopulating cells can engraft NOD/SCID-β₂M^{-/-} mice allows the development of an assay to detect each of the cell types.

(a) STRC-M

A hallmark of the short term repopulating cell-myeloid (STRC-M) is the large and rapid but transient burst of erythroid cells they produce in the first 3 weeks post-transplant, although analyses of oligoclonally repopulated mice showed that these cells also consistently produced detectable numbers of granulocytes and megakaryocytes (but not lymphoid cells). Most STRC-M from normal adult human BM were shown to express CD38 and could be rapidly amplified (>10-fold) in short term culture. They were also present at high levels in the CD34⁺ compartment of mPB and were detectable but at relatively reduced levels in CB. These features indicate a stage of stem cell differentiation characterized by a lack of self-renewal activity and lymphopoietic potential, most likely analogous to murine day 9-12 CFU-S (31,32) and the recently described common myeloid progenitor (13).

Accordingly, the present invention provides a short term repopulating human cell that can produce myeloid cells in NOD/SCID-β₂M^{-/-} mice. These cells are termed STRC-M herein. The STRC-M cells are characterized by the rapid production of erythroid cells produced in the first three weeks post-transplant in the mouse. The STRC-M show consistent myeloid (i.e., granulocytes and megakaryocytes) engraftment (3-8 weeks) but no lymphoid generation. The STRC-M are CD34⁺ and CD38⁺.

The present invention includes a method of detecting a short term repopulating human cell that can produce myeloid cells in NOD/SCID-β₂M^{-/-} mice comprising (a) transplanting human hematopoietic cells in a NOD/SCID-β₂M^{-/-} mouse and (b) detecting human erythroid cells at approximately three weeks post-transplant.

(b) STRC-ML

The evidence for the short term repopulating cell-lympho-myeloid cells (STRC-ML) (which represent a population distinct from the lympho-myeloid cells that engraft NOD/SCID mice) is based on a different set of observations. The first of these indicated a difference in the kinetics of human lympho-myeloid engraftment of NOD/SCID-β₂M^{-/-} and NOD/SCID mice which reached a much higher peak after 6-8 weeks and then declined more rapidly in the NOD/SCID-β₂M^{-/-} hosts so that the total level of engraftment in the 2 strains was increasingly similar by 13 weeks. Further evidence that these differential kinetics reflect the superimposed activity in the NOD/SCID-β₂M^{-/-} mice of STRC as well as long term repopulating cells (LTRC) with unrestricted differential potential was provided by the demonstration that cells able to engraft NOD/SCID mice do not home to the marrow of NOD/SCID-β₂M^{-/-} mice at a higher efficiency and their subsequent amplification is also not enhanced in NOD/SCID-β₂M^{-/-} mice. Finally, the inventors showed the ability of STRC-ML to engraft NOD/SCID-β₂M^{-/-} mice is not altered when these cells transit S/G₂/M. This contrasts dramatically with the behavior of the lympho-myeloid cells that repopulate NOD/SCID mice whose engrafting activity is severely compromised when they proliferate (29,33). Parallel differences between short and prolonged engraftment durability and low and high sensitivity to cell cycle progression have been reported for murine repopulating cells (27) Further studies will be required to determine whether human STRC-ML and LTRC can also be phenotypically separated and related to corresponding subsets of murine stem cells. Such information would also facilitate comparisons between the types of cells able to engraft NOD/SCID-β₂M^{-/-} mice and fetal sheep and the extent and durability of lympho-myeloid cell production achievable from each in these two xenotransplant models. It is interesting to note that recent clonal analyses of gene-marked autografts in nonhuman primates have revealed exclusively myeloid progeny of marked cells (both erythroid and granulopoietic) up to 24 weeks post-transplant and only after that time did lympho-myeloid clones become detectable (34).

Accordingly, the present invention provides a short term repopulating human cell that can produce myeloid and lymphoid cells in NOD/SCID-β₂M^{-/-} mice. These cells are termed STRC-ML herein. The STRC-ML cells are characterized by a transient burst in lymphoid and myeloid cell production that peeks at 6-8 weeks. The cells are further characterized in that they maintain their engraftment potential when they proliferate. These cells can therefore be expanded in vitro to facilitate in vivo engraftment.

The present invention also includes a method of detecting a short term repopulating human cell that can produce myeloid and lymphoid cells in NOD/SCID-β₂M^{-/-} mice comprising (a) transplanting human hematopoietic cells in a NOD/SCID-β₂M^{-/-} mouse and (b) detecting human myeloid and lymphoid cells at approximately six to eight weeks post-transplant.

The presence of the short term repopulating cells, STRC-M and STRC-ML, may be detected using the methods described in Example 1. Briefly, the NOD/SCID-β₂M^{-/-} mice are irradiated prior to injection with a human hematopoietic cell sample. The hematopoietic cell sample can be from any source including peripheral blood, bone marrow and cord blood as well as tissues containing hematopoietic cells such as lymphoid tissue, epithelia, thymus, liver, spleen, lymph node tissue, cancerous tissue or fetal tissue including fetal liver or cells derived from embryonic stem cells.

The presence of the STRC-M are detected in the mouse at approximately 3 weeks and the STRC-ML at approximately 6-8 weeks post transplant The cells are preferably detected in the bone marrow although other samples may be used. The bone marrow may be obtained from femora or tibiae. The cells can be detected using a variety of techniques including FACS analysis and immunocytochemical staining. The STRC-M produce erythroid as well as some granulocytes and megakaryocytes but not lymphoid cells at 3 weeks post transplant. These cells can be detected by staining for glycophorin A⁺ or CD71⁺ (erythroid cells) or CD41⁺ (megakaryocytes) or CD15⁺/66b⁺ (granulocytes) cells in a sample collected from the mouse at approximately 3 weeks post transplant. The STRC-ML produce myeloid and lymphoid cells at about 6 to 8 weeks post transplant. These cells can be detected by staining for CD34⁻CD19/20⁺ cells (lymphoid cells) and glycophorin A⁺ or CD41⁺ or CD15/66b⁺ (myeloid cells) cells in a sample collected from the mouse at approximately 6 to 8 weeks post transplant.

The frequency of short term repopulating cells in a suspension of human hematopoietic cells (mobilized peripheral blood, bone marrow or cord blood) can be determined by limiting dilution in the assay of the invention. NOD/SCID-β₂M^{-/-} mice are engrafted with decreasing numbers of cells from the sample to be tested. At some point in the titration there will be insufficient short term repopulating cells to produce detectable human cells in the bone marrow harvested from both femora and tibiae.

The degree of engraftment of human cells measured in the bone marrow of a NOD/SCID-β₂M^{-/-} mouse using the assay of the invention is an indication of the relative frequency of short term repopulating cells present in the human cells injected into the mouse. Therefore, it can be used to compare two different human cell suspensions, exposed to different treatments provided the total number of human test cells infused per mouse remains constant.

### II. Uses

The inventors have shown that NOD/SCID-β₂M^{-/-} mice support a broader range of transplantable human cells than NOD/SCID mice including the STRC-M and STRC-ML cells described above. This enables the development of assays for detecting STRC-M and STRC-ML that are useful in assessing the engraftment potential of human hematopoietic cells, testing the toxicity of various drugs and in assessing the effects of ex vivo storage and processing on hematopoietic transplant grafts. In additiona, the use of these assays in combination with gene marking studies and to analyze leukemic populations should help to identify the molecular mechanisms that distinguish early stages of normal and leukemic stem cell differentiation.

(a) Evaluation of Engraftment Potential and Kinetics of a Hematopoietic Transplant Graft

Hematopoietic cell transplant recipients are often heavily pre-treated such that the hematopoietic potential of their bone marrow or their ability to mobilize primitive hematopoietic cells into the periphery during stem cell mobilization may be reduced. The hematopoietic potential of cord blood harvests also varies greatly depending on the level of contamination with maternal blood. An indication of the repopulating potential of these grafts is crucial in determining whether to proceed with the transplant and how many cells to give. There are certain cell phenotypes indicative of the presence of primitive cells but these do not replace the functional measure of in vivo repopulation which is only offered by animal models. The assay of the invention can be used to measure the short term repopulating potential of a hematopoietic cell harvest.

Accordingly, the present invention provides a method of assessing the short term repopulating potential of human hematopoietic cells comprising:

(a) administering the human hematopoietic cells to a NOD/SCID-β₂M^{-/-} mouse;

(b) obtaining a sample from the mouse at approximately 3 weeks after step (a);

(c) assaying the sample for human short term repopulating cells-myeloid (STRC-M) wherein the presence of STRC-M indicates that the human hematopoietic cells have short term repopulating potential.

The presence of STRC-M in the initial sample can be assayed by detecting human erythroid cells that are produced at approximately 3 weeks post transplant. Human granulocytes and megakaryocytes may also be detected as well as the absence of lymphoid cells. Methods for detecting the particular cell types are well known in the art and are described previously and in Example 1.

The present invention also provides a method of assessing the short term repopulating potential of human hematopoietic cells comprising:

(a) administering the human hematopoietic cells to a NOD/SCID-β₂M^{-/-} mouse;

(b) obtaining a sample from the mouse at approximately 6-8 weeks after step (a);

(c) assaying the sample for human short term repopulating cells-lympho-myeloid (STRC-ML) wherein the presence of STRC-ML indicates that the human hematopoietic cells have short term repopulating potential.

The presence of STRC-ML in the initial sample can be assayed by detecting human myeloid and lymphoid cells that are produced in the mouse at approximately 6 to 8 weeks post transplant. Methods for detecting myeloid and lymphoid cells are well known in the art and are described previously and in Example 1.

(b) Toxicity Testing of Potential Drugs

The hematopoietic system is very sensitive to the toxic effects of irradiation and chemotherapy. Effects on hematopoiesis may severely limit the usefulness and safety of a drug. The functional effects of new drugs on hematopoietic cells must be studied in in vitro assays or animal models. The method of the invention is the first in vivo assay for human short term repopulating cells. Drug toxicity tests done before clinical trials will involve exposure of human hematopoietic cells in vitro to the drug and the cells then tested in the assay of the invention (STRC-M, STRC-ML). Once the drug is administered to patients the effect on the patients hematopoietic cells can be followed by harvesting a bone marrow sample and running this sample in the assay of the invention.

The present invention further provides a method of assessing the toxicity of a drug on human hematopoietic cells comprising:

(a) exposing human hematopoietic cells to the drug;

(b) administering the cells from (a) to a NOD/SCID-β₂M^{-/-} mouse;

(c) obtaining a sample from the mouse at approximately 3 weeks after step (b);

(d) assaying the sample for human short term repopulating cells-myeloid (STRC-M) wherein the presence of STRC-M at levels equal to that of untreated cells indicates that the drug is not toxic to these cells.

The present invention also provides a method of assessing the toxicity of a drug on human hematopoietic cells comprising:

(a) exposing human hematopoietic cells to the drug;

(b) administering the cells from (a) to a NOD/SCID-β₂M^{-/-} mouse;

(c) obtaining a sample from the mouse at approximately 6 to 8 weeks after step (b);

(d) assaying the sample for human short term repopulating cells-lympho-myeloid (STRC-ML) wherein the presence of STRC-ML at levels equal to that of untreated cells indicates that the drug is not toxic to these cells.

The term “untreated cells” means human hematopoietic cells that have not been exposed to the drug. The untreated cells will be from the same source as the treated cells and will be subjected to the same treatment as the treated cells, except for exposure to the drug.

(c) Assessment of the Affect of ex vivo Storage and Processing of Hematopoietic Transplant Grafts

Clinical transplantation of hematopoietic cells involves harvesting, storing and potentially separating the cells in the transplant graft. All these ex vivo graft processing techniques are constantly being upgraded and expanded. Any change in technique or processing equipment requires extensive testing to ensure the repopulating potential of the graft has not been compromised. The method of the invention offers a way to test for any effect on short term repopulating potential. Samples of bone marrow, mobilized peripheral blood and cord blood can be processed with the old and new protocols and then assessed using the method of the invention.

The present invention provides a method of assessing the viability of a human hematopoietic cell sample comprising:

(a) administering the human hematopoietic cells to a NOD/SCID-β₂M^{-/-} mouse;

(b) obtaining a sample from the mouse at approximately 3 weeks after step (a);

(c) assaying the sample for human short term repopulating cells-myeloid (STRC-M) wherein the presence of STRC-M indicates that the sample has viable short term repopulating cells.

The present invention also provides a method of assessing the short term repopulating potential of human hematopoietic cells comprising:

(a) administering the human hematopoietic cells to a NOD/SCID-β₂M^{-/-} mouse;

(b) obtaining a sample from the mouse at approximately 6 to 8 weeks after step (a);

(c) assaying the sample for human short term repopulating cells-lympho-myeloid (STRC-ML) wherein the presence of STRC-ML indicates that the sample has viable short term repopulating cells.

The following non-limiting examples are illustrative of the present invention:

### EXAMPLES

### Example 1

### Xenotransplantation Assay

NOD/LtSz-scid/scid β₂M^{-/-} mice were irradiated at 8-10 weeks of age with 350 cGy of ¹³⁷Cs x-rays and thereafter received acidified water containing 100 mg/L ciprofloxacine (Bayer, Leverkusen, Germany). Test cells were injected intravenously with 10⁶ irradiated (15 Gy) normal human BM cells as carrier cells within a few hours after the mice were irradiated. The presence of human cells in the BM of mice was determined by FACS analysis after first blocking Fc receptors with human serum and an anti-mouse Fc receptor antibody 2.4G2 (from Pharmingen, Mountainview, Calif.) followed by staining with monoclonal antibodies against human CD34 (8G12), CD71 (OKT9), glycophorin A (10F7, kindly provided by P. M. Lansdorp), CD15, CD19, CD20, CD45 (from Becton Dickinson), and CD41a and CD66b (from Pharmacia Biotech, Baie d-Urfe, PQ). Levels of nonspecific staining were established by parallel analyses of cells incubated with irrelevant isotype-matched control antibodies labeled with the same fluorochromes. Positive events were counted using gates set to exclude >99.99% events in the negative control analyses. Poisson statistics and the method of maximum likelihood was used to calculate frequencies of human repopulating cells from proportions of negative mice within one or a series of similar experiments. Specific details on the protocol are provided below.

### Short Term Repopulating Cell Assay—the Protocol

1. Acidified water (pH 3.0) containing antibiotics should be provided to NOD/SCID-β₂M^{-/-} mice, ad libitum 2-7 days prior to irradiation and for 4-6 weeks following transplantation.

2. Sublethally irradiate NOD/SCID-β₂M^{-/-} recipients by exposure to 350 cGy of total body γ-irradiation administered in a single dose at <250 cGy/min. Irradiate sufficient animals to allow 3-4 groups of 4-8 animals per group.

3. Irradiate normal human bone marrow (BM) cells with 1500 cGy for use as carrier cells.

4. Prepare cell mixtures in Iscove's modified Dulbecco's medium (IMDM)/2% Fetal Calf Serum (FCS) such that 0.25 ml contains the desired dose of test cells and 10⁶ carrier cells. Appropriate test cell doses for limiting-dilution analysis are as follows: 10⁵-10⁶ unseparated mononuclear cells from bone marrow, cord blood or mobilized peripheral blood; 10³-10⁴ lineage depleted cells from bone marrow, cord blood or mobilized blood

5. Inject 0.25 ml of each cell mixture intravenously into the tail veins of irradiated NOD/SCID-β₂M^{-/-} mice. Recipients should be injected within a few hours following irradiation.

6. STRC-M are read out at 3 weeks and STRC-ML are read out at 6-8 weeks. Collect BM cells from both femora and tibiae into 5 ml of cold Hank's balanced salt solution plus 2% fetal bovine serum (HF)/5% Human Serum (HS) using a sterile 21-gauge needle and a 3 ml syringe. Count viable nucleated BM cells.

7. Pellet BM cells. Lyse erythrocytes by resuspending cells in ˜3 ml of ammonium chloride red cell lysing solution and incubate 5 mins on ice. Wash cells once with cold HF buffer and decant supernatant. Finally resuspend cells in 5 ml HF/5% HS. Note that lysis of red blood cells is not required if they are excluded by gating during FACS analysis.

8. Dispense 0.2 ml cells into each of five FACS tubes and add 2.4G2 monoclonal antibody to a final concentration of 3 mg/ml. This facilitates blocking of F_{c} receptors and prevents non-specific binding of subsequent antibodies. Incubate cells for 10 min at 4° C. It is not necessary to wash cells prior to proceeding to step 9.

9. Add the following antibodies to the 6 sample tubes:

a) Nothing; cells in HF/PI only (unstained control).

b) IgG-FITC and IgG-PE (isotype controls).

c) IgM-FITC and IgG-PE (isotype controls).

d) CD34-FITC, CD-19-PE and CD20-PE.

e) CD15-FITC, CD66b-FITC, CD41-PE and Glycophorin A˜PE.

f) CD41-PE and Glycophorin A-FITC

Tubes a, b and c are used to establish threshold settings. All anti-human monoclonal antibodies must be titrated using human cells and tested for non-reactivity against BM cells from naive NOD/SCID-β₂M^{-/-} mice.

10. Protect all tubes from light and incubate for 30 min on ice.

11. Wash all samples twice with ˜3 ml HF and finally resuspend cells in 0.2 mL HF/PI for flow cytometric analysis.

12. Establish quadrant or region parameters for negative cells based on the background levels of fluorescence observed with PI negative cells stained with FITC-and PE-labeled isotype-matched control antibodies. Positive cells are defined as those exhibiting a fluorescence that exceeds 99.98% of that obtained with negative controls labeled with the same fluorochromes. Score mice as positive for STRC-M if there are 5 or greater Glycophorin A positive or CD41⁺ or CD15/66b⁺ cells per 2×10⁴ PI⁻ cells. Score mice as positive for STRC-ML if there are 5 or greater CD34⁻CD19/20⁺ and 5 or greater glycophorin A positive or CD41⁺ or CD15/66b⁺ cells per 2×10⁴ PI⁻ cells. Because this threshold (0.025% engraftment) is very near to the limit of sensitivity of FACS, it is absolutely critical that negative and isotype control samples are clean. If technical problems or proficiency with flow cytometric analysis compromise these controls, investigators may need to define higher levels of engraftment (e.g. ≧0.5%) for the human CRU assay. Note: Immunocytochemical staining can be used to detect engrafted human cells in the method of the invention.

### Example 2

### Human Lin− BM Cells Engraft NOD/SCID-β₂M^{-/-} and NOD/SCID Mice With Different Kinetics

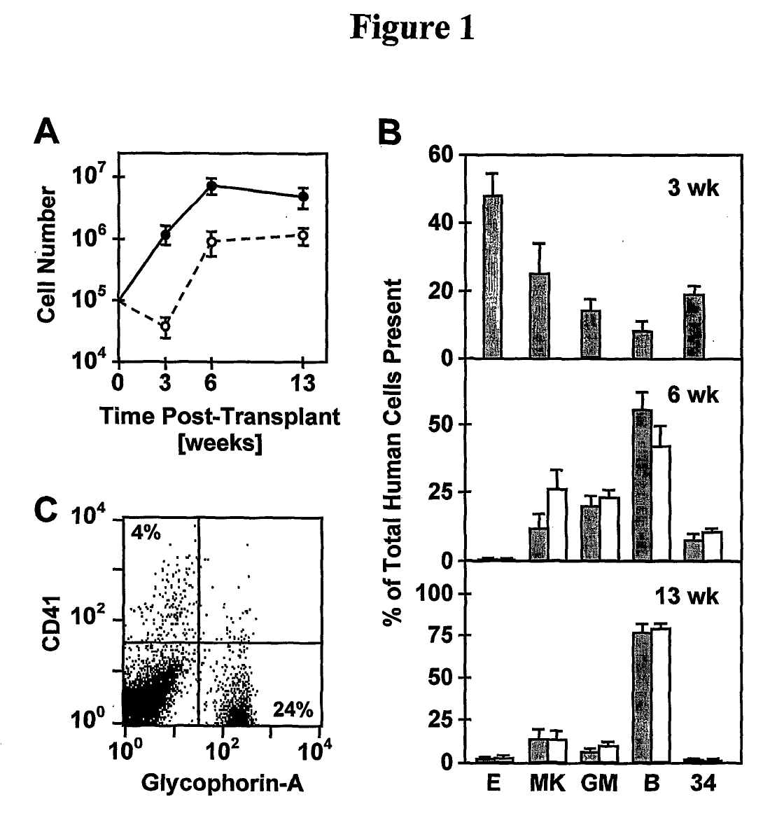

FIG. 1 shows the different engraftment kinetics of human cells in NOD/SCID-β₂M^{-/-} mice and NOD/SCID mice. Groups of recipients were sacrificed 3, 6, and 13 weeks after transplantation and the types and numbers of human cells present in the bone marrow determined by FACS analysis. FIG. 1A: Total human CD45/71⁺ cells in NOD/SCID-β₂M^{-/-} mice (solid symbols, 13-14 mice/point) and NOD/SCID mice (open symbols, 15-16 mice/point) were calculated from data pooled from 2 independent experiments. FIG. 1B: Production of particular hematopoietic lineages shown as a proportion of the total human CD45/71⁺ cells present after different periods in NOD/SCID-β₂M^{-/-} (solid bars) and NOD/SCID mice (open bars, same experiments as Panel A). FIG. 1C: Representative FACS profile of cells harvested from the BM of a NOD/SCID-β₂M^{-/-} mouse 3 weeks after transplantation of 2.5×10⁵ human lin⁻ BM cells. Note the high number of human erythroid (glycophorin A⁺) and megakaryocytic (CD41⁺) cells.

As shown in FIG. 1A, when decreasing numbers of lin⁻ cells isolated from normal adult BM were transplanted into parallel groups of sublethally irradiated NOD/SCID-β₂M^{-/-} and NOD/SCID mice more human cells were present in the NOD/SCID-β₂M^{-/-} mice at all times analyzed up to 13 weeks post-transplant (p<0.03). However, the difference between the levels of engraftment obtained in the two mouse strains was most pronounced (˜30-fold) at the 3 week time point. By 6 weeks this difference had decreased to 8-fold and by 13 weeks was only 4-fold. The large difference seen at 3 weeks post-transplant was due primarily to the presence in the NOD/SCID-β₂M^{-/-} mice of a large population of human glycophorin A⁺ erythroid cells, CD41⁺ megakaryocytic cells and CD15/66b⁺ granulopoietic cells (FIGS. 1B and C). In addition, human CD34⁺ cells and occasional CD19/20⁺ B-lymphoid cells were seen. At later times, the lineage distribution of hematopoietic cell types in both mouse strains was similar with B-lymphoid cells having become the predominant cell type and maturing erythroid cells being rarely seen.

### Example 3

### Different Types of Human Cells Engraft NOD/SCID-β₂ M^{-/-} Mice and NOD/SCID Mice

Example 3 demonstrates whether the initially high but transient output of human erythroid and megakaryocytic cells seen exclusively in the BM of NOD/SCID-β₂M^{-/-} mice were produced by a specific subtype of human progenitor. As a first approach, Poisson statistics were used to calculate the frequency of CD34⁺ cells in the injected BM that were able to repopulate the marrow of each strain of mouse for different periods of time. For this comparison, a repopulating cell was defined as any cell that produced ≧10 human cells expressing either CD45 and/or CD71 per 2×10⁴ viable cells analyzed. As shown in Table 1, the frequency of 3 week repopulating cells measured using NOD/SCID-β₂M^{-/-} hosts was ˜30-fold higher than the frequency of cells able to repopulate NOD/SCID mice within the same early time frame (p<0.03), i.e. a factor similar to that seen when total engraftment levels in the two recipient genotypes were compared. Moreover, approximately half of the 3 week-engrafted NOD/SCID-β₂M^{-/-} mice that had been injected with limiting numbers of any type of human repopulating cell (on average <4 contained human myeloid cells (erythroid, megakaryocytic and granulopoietic) exclusively (i.e., no lymphoid cells)). The limiting dilution analysis also showed that the human BM cells that regenerate the mature cells seen in NOD/SCID-β₂M^{-/-} mice at later times were also much more prevalent than those able to reconstitute NOD/SCID mice (p<0.03), although most of these displayed both lymphoid and myeloid potential.

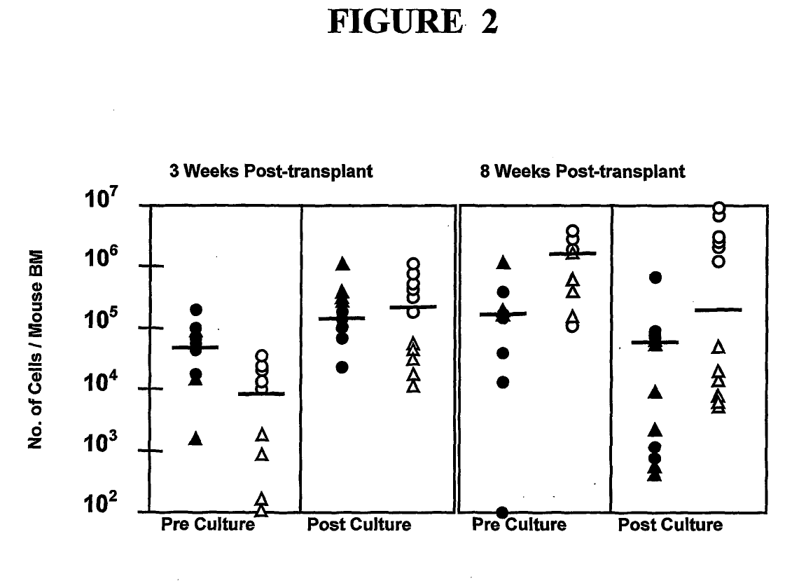

A second series of experiments were then undertaken to determine whether the progenitors of these different progeny populations could be distinguished phenotypically. Accordingly, the CD38⁺ and CD38⁻ subsets of lin⁻ CD34⁺ adult marrow cells were isolated by FACS and then assessed for their 3 and 8 week repopulating activity in NOD/SCID-β₂M^{-/-} mice. FIG. 2 illustrates that CD34⁺CD38⁺ cells (open symbols) in adult BM show an initially greater ability than CD34⁺CD38⁻ cells (solid symbols) to engraft NOD/SCID-β₂M^{-/-} mice for 3 weeks, but CD34⁺CD38⁻ cells have an equivalent ability to produce this activity in 5 day expansion cultures. In contrast, CD34⁺CD38⁺ cells contribute much less to the 8 week engraftment of NOD/SCID-β₂M^{-/-} mice and show a parallel decline in this activity after 5 days in culture. Each symbol corresponds to the level of engraftment seen in an individual mouse originally injected with the yield of CD38⁺ or CD38⁻ cells obtained from a starting equivalent of 10⁵ CD34⁺ cells either directly (Pre culture) or after 5 days of culture with FL, SF, IL-3, IL-6 and G-CSF (Post culture).

As shown in FIG. 2, the CD38⁺ subset was responsible for most of the 3 week repopulating activity. Conversely, most of the human cells present after 8 weeks were generated from CD34⁺CD38⁻ cells. Limiting dilution analysis of the frequency of 3 week repopulating cells yielded a value of 1 per 1.3×10⁵ (with a range defined by ±SEM of 1 per 1 to 1.7×10⁵) CD34⁺/CD38⁺ cells. CD34⁺/CD38⁺ cells thus accounted for ˜85% of all the 3 week repopulating activity in the CD34⁺ population.

The inability of human STRC to engraft NOD/SCID mice in spite of equivalent engraftment and self-renewal in NOD/SCID-β₂M^{-/-} mice of LTRC suggests an early change in differentiating human stem cell populations that renders them sensitive to rejection mechanisms that are eliminated by introduction of the β₂M null mutation into the NOD/SCID genotype. Both mice lack B and T cells and hemolytic complement, but differ in the extent of NK cell activity they possess (23). In NOD/SCID mice NK cells are reduced but not absent, whereas in NOD/SCID-β₂M^{-/-} mice NK cell activity is undetectable. It is, therefore, inviting to speculate that the mechanism underlying the differential engraftment of human STRC in these 2 mouse strains may involve parameters that increase their ability to be recognized or killed by foreign NK cells. Such an explanation, if validated, would predict that allogeneic clinical transplants might also result in delayed hematologic recovery due to impaired engraftment of the STRC they contain.

On the other hand, the inability of human STRC to engraft NOD/SCID mice enables human LTRC to be detected and quantified in these recipients with greater specificity at early times post-transplant without the need to undertake a pre-enrichment or serial transplant step. This feature has obvious practical advantages and should allow further analysis of the most primitive types of human stem cell populations. For example, it would be anticipated from the findings reported here that human CD34⁻ stem cells would also not engraft NOD/SCID-β₂M^{-/-} mice any more efficiently than NOD/SCID hosts.

Some murine LTRC can start to produce mature blood cells almost as quickly after transplantation as those that do not have durable engraftment abilities (11,35). Nevertheless, recovery rates of peripheral blood neutrophil and platelet counts relative to one another in patients can be highly variable and, in some cases, recovery of both can be very protracted. Moreover, differences in the average rate of recovery of the blood counts seen with different types of transplant do not correlate with their content of NOD/SCID repopulating cells. In particular, the frequency of NOD/SCID repopulating cells in mPB has been found to be 15- and a 120-fold lower than in BM or CB (4), whereas even saturating doses of BM fail to elicit as rapid recovery rates in patients as transplants of mPB (7). The inventors have shown that both STRC-M and STRC-ML activities are markedly elevated in mPB by comparison to their published LTRC content which is more consistent with their rapid engraftment kinetics in patients.

### Example 4

### The Seeding Efficiency and Subsequent Expansion in vivo of Cells That Repopulate NOD/SCID Mice for 6 Weeks is Similar in NOD/SCID-β₂M^{-/-} and NOD/SCID Mice

This example illustrates that NOD/SCID-β₂M^{-/-} mice are not simply more efficient in their ability to support the engraftment of the lympho-myeloid human cells that repopulate NOD/SCID mice. For this, the inventors first compared the seeding efficiency of human NOD/SCID repopulating cells in NOD/SCID-β₂M^{-/-} mice and NOD/SCID mice. Because of the low frequency of these cells in adult human BM (Table 1 and 4,25) and the relatively higher numbers in human fetal liver, the latter source was used for these particular experiments. Accordingly, 2×10⁷ low density fetal liver cells were injected into groups of NOD/SCID-β₂M^{-/-} mice and NOD/SCID mice, and then 24 hours later, the BM cells were harvested and transplanted into groups of secondary NOD/SCID recipients. Six weeks later, the proportion of the secondary mice containing both human lymphoid (CD34⁻CD19/20⁺) and myeloid (CD15/66b⁺) cells was determined and the number of NOD/SCID lympho-myeloid repopulating cells that had seeded into the marrow of the primary recipients within the first 24 hours then calculated. Separate determination of the number of 6 week lympho-myeloid NOD/SCID repopulating cells injected into the primary mice was made by limiting dilution analysis of a second set of primary NOD/SCID mice who were transplanted with smaller aliquots of the same human fetal liver cells and then assessed for the presence of human lymphoid and myeloid cells in their marrow 6 weeks later. Using this number, the efficiency of seeding into the BM of primary NOD/SCID-β₂M^{-/-} and NOD/SCID mice was calculated from the pooled data of 3 experiments to be 1.4% and 2.5%, respectively.

In a further series of experiments, the inventors compared the ability of 6 week lympho-myeloid NOD/SCID repopulating cells to expand their numbers after transplantation of human low density fetal liver cells into the two genotypes of mice (˜10⁵ CD34⁺ cells per mouse) by secondary transplants into NOD/SCID mice 4 weeks later. The frequency and hence the number of regenerated cells with 6 week lympho-myeloid NOD/SCID repopulating potential was again found to be similar for NOD/SCID-β₂M^{-/-} or NOD/SCID primary hosts (1 per 2.6×10⁴ and 1 per 3.3×10⁴ CD34⁺ cells injected into primary recipients, p>0.05). The self-renewal behavior of human stem cells that engraft NOD/SCID mice thus appears to be duplicated but not enhanced in NOD/SCID-β₂M^{-/-} mice. This result, together with the seeding efficiency data, indicates no advantage in NOD/SCID-β₂M^{-/-} mice of the type of human stem cells that repopulate NOD/SCID mice. Therefore, the enhanced human multi-lineage engraftment seen in NOD/SCID-β₂M^{-/-} mice up to even 13 weeks post-transplant is more likely indicative of a second category of short term repopulating human cells which have lympho-myeloid differentiation potential, but are unable to repopulate NOD/SCID mice.

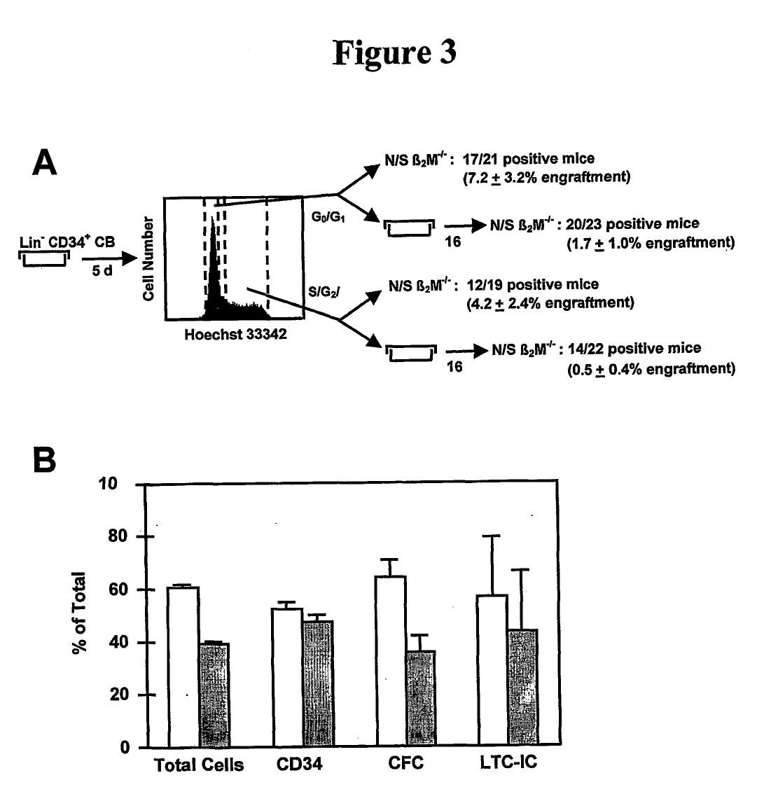

Additional evidence to support this conclusion was provided by experiments with human repopulating cells that had been stimulated to proliferate in vitro. Previous studies have shown that murine cells with short and long term repopulating activity differ in their ability to retain this activity in syngeneic hosts as they progress through the cell cycle; the engraftment ability of short term repopulating cells being little affected, (26,27) in contrast to long term repopulating cells which are severely compromised during their passage through S/G₂/M. FIG. 3 shows that G₀/G₁ and S/G₂/M cells from 5 day expansion cultures of human CB cells show equivalent distributions of repopulating activity in NOD/SCID-β₂M^{-/-} mice (A) and progenitor numbers detected in vitro (B). CD34⁺ CB cells were cultured for 5 days in serum-free medium supplemented with SF, FL, IL-3, IL-6, and G-CSF. G₀/G₁ and S/G₂/M cells were then isolated after DNA staining with Hoechst 33342. Approximately half of the fractionated cells were transplanted in NOD/SCIDβ₂M^{-/-} mice immediately after their isolation. Equal portions were first cultured for an additional 16 hours before being transplanted. There was no correlation between the proportion of engrafted mice and the percentage of cells in any cell cycle stage. B: Proportion of total cells, CD34⁺ cells, CFC, and LTC-IC in G₀/G₁ (open bars) and S/G₂/M (solid bars) measured after the first 5 days of culture in aliquots from the same experiments. All values shown are the mean ±SEM of data pooled from 3 experiments.

The inventors have recently shown that human CB cells in S/G₂/M also show a lack of repopulating activity when transplanted into NOD/SCID mice (29). As shown in FIG. 3, no such deficiency was evident when proliferating CB cells were assessed for their ability to engraft NOD/SCID-β₂M^{-/-} mice for 6 weeks and these showed the same distribution between G₀/G₁ and S/G₂/M as seen for other endpoints of primitive cell activity (both in terms of the relative proportions of engrafted mice (56% vs 44%) and the levels of engraftment attained (7.2% vs 4.2%). Moreover, further culture of the separated G₀/G₁ and S/G₂/M cells did not differentially alter the NOD/SCID-β₂M^{-/-} repopulating activity exhibited by their progeny assessed one day later.

### Example 5

### The Short and Long Term Repopulating Activities of Different Human Tissues Vary Independently

Transplants of human cells from different sources are known to be associated with different clinical engraftment kinetics. Moreover, these do not correlate with their content of 6-8 week NOD/SCID repopulating cells. Thus, by comparison to normal adult BM, mPB samples contain a relatively low frequency of NOD/SCID repopulating cells, (4,30) in spite of the fact that their clinical use is typically associated with more rapid hematologic recovery (7). This situation is just the opposite for CB (4). It was, therefore, of interest to compare the levels of engraftment obtained after 3 and 6-8 weeks in NOD/SCID-β₂M^{-/-} mice transplanted with CD34⁺ cell-enriched populations isolated from these 3 different sources of cells. As shown in Table 2, in all groups, the level of engraftment was higher at the later time point although the differences between 3 and 6-8 weeks were specific for each source of cells. Moreover, the progeny seen after 3 weeks were again primarily erythroid (glycophorin A⁺) whereas after 6-8 weeks all mice contained both lymphoid and myeloid cells.

### Example 6

### Selective Expansion of Human Stem Cells with Short-Term Repopulating Activity in Short-Term Cultures of Human Marrow

Currently, much effort is focused on the identification of culture conditions that would allow the pace of hematologic recovery in transplant recipients to be accelerated. To determine the extent to which human cells with rapid repopulating activity are expanded in vitro and to characterize the phenotype of their precursors, FACS-purified CD34⁺CD38⁻ and CD34⁺CD38⁺ were isolated from adult bone marrow lin⁻ cells, and then aliquots were transplanted into NOD/SCID-β₂M^{-/-} mice before and after being maintained in serum-free expansion cultures for 5 days with FL, SF, IL-3, IL-6 and G-CSF. In both of two such experiments performed, a several fold increase in early (3 week) engrafting activity was obtained . This increase was >20-fold from the initially CD34⁺CD38⁻ cell fraction although after 5 days, the early engrafting activity of the cells generated from the CD38⁺ and CD38⁻ subsets was approximately equal (FIG. 2). In contrast, the level of engraftment achieved after 8 weeks from the cultured bone marrow cells was maintained in one experiment and declined ˜30-fold in the other, regardless of the phenotype of the cells originally cultured.

### Example 7

### Evidence of Early Transient Engraftment of NOD/SCID-β₂ Microglobulin Null Mice With Neoplastic Cells From Myelodysplastic Syndrome Patients

Myelodysplastic syndromes (MDS) are clonal disorders usually involving all myeloid hematopoietic cell lineages and a reduction in cells with in vitro CFC or LTC-IC activity. The inventors have now assessed the ability of sublethally irradiated immunodeficient mice to be engrafted with cells from MDS patients using NOD/SCID-β₂ microglobulin null (NOD/SCID-β₂M^{-/-}) mice and NOD/SCID-β₂M^{-/-} mice engineered transgenically to produce human Steel Factor, IL-3 and GM-CSF (serum levels of 1-4 ng/mL) as recipients. Mice were injected IV with 4-15×10⁶ low density bone marrow (BM) or blood cells from 4 patients with MDS (1RARS, 1 CMML, 2 RAEBT) and then serial BM aspirations were performed 3 to 8 wk post-transplant and engraftment assessed by flow cytometry. Human CD45/71⁺ cells were detected in 85% of all mice (28 of 33) at levels ranging from 0.1 to 70% 3 wk after injection, with no obvious difference in either the proportion of positive mice, or the levels of engraftment between the 2 types of recipient (eg, average 4% vs 14% human CD45/71⁺ cells at 3 wk). In recipients of cells from 3 patients, the human population was almost exclusively (91%) CD15/66b⁺. Only in a few cases could occasional human CD34⁺ cells be detected. By 4 wk, the proportion of human CD45/71⁺ cells decreased consistently and dramatically (10-fold) and there was no change in the phenotype of the human cells present. By wk 5, all evidence of early engraftment disappeared. In recipients of cells from 2 patients, human cells did not reappear over the next 3 wk. However, at 7 wk post-transplant, 4 of 6 mice injected with cells from the other 2 patients contained both CD15/66b⁺ and CD19/20⁺ human cells (0.33% and 0.25%). In the other 2 mice (one for each patient), CD15/66b⁺ cells were the only human subset present (2% of total). FISH analysis of FACS-sorted human CD45/71⁺ cells obtained from chimeric recipients of one patient's cells at both 3 and 7 wk post-transplant showed the +8 cytogenetic abnormality seen in the original BM population to be present at a similar frequency (6%). Although the study involves a small number of patients, the consistent detection (especially early after injection) of a predominantly myeloid (CD 15/66b⁺) population that included cytogenetically abnormal elements suggests that certain types of human MDS precursors are able to home into the BM of mice and differentiate. These findings provide a starting point for future studies of the properties of transplantable normal and neoplastic populations in patients with MDS.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Frequencies of repopulating cells in human BM detected in NOD/SCID-
β₂M^{-/-} and NOD/SCID mice at different time points after injection.

| Frequency of repopulating cells* | | | Proportion of positive mice** | | | | | |
|---|---|---|---|---|---|---|---|---|
| Time post- | NOD/ | | NOD/SCID- | | | NOD/ | | |
| transplant | SCID- | NOD/ | β₂M^{-/-} | | | SCID | | |
| (wk) | β₂M^{-/-} | SCID | M⁺L⁺ | M⁺L⁻ | M⁻L⁺ | M⁺L⁺ | M⁺L⁻ | M⁻L⁺ |
| 3 | 1 per 6.2 × 10³ | 1 per 1.6 × 10⁵ | 5/14 | 6/14*** | 0/14 | 1/15 | 6/15 | 0/15 |
| | (4.2 − 9.2 × 10³⁾ | (1.1 − 2.2 × 10⁵⁾ | | | | | | |
| 6 | 1 per 8.7 × 10³ | 1 per 5.3 × 10⁴ | 8/15 | 0/15 | 2/15 | 7/15 | 0/15 | 0/15 |
| | (6.0 − 13 × 10³⁾ | (3.5 − 7.9 × 10⁴⁾ | | | | | | |
| 13 | 1 per 6.7 × 10³ | 1 per 1.1 × 10⁵ | 5/16 | 0/16 | 5/16 | 4/15 | 0/15 | 4/15 |
| | (4.5 − 9.9 × 10³⁾ | (0.7 − 1.6 × 10⁵⁾ | | | | | | |

*Cells able to generate ≧10 human CD45/71⁺ cells per 2 × 10⁴ PI⁻ cells analyzed per CD34⁺ cells in the lin⁻ population injected.
**From mice injected with a dose of lin⁻ cells calculated to contain less than 4 cells able to generate delectable numbers of any kind of human progeny. Note that the NOD/SCID mice received on average >10-fold more human cells than the NOD/SCID-β₂M^{-/-} mice.
***The human lineages represented in these mice were: 39 ± 9% erythroid (glycophorin A⁺ ) cells, 37 ± 20% megakaryocytic (CD41⁺) cells and 16 ± 4% granulopoietic (CD15/66b⁺) cells.

TABLE 2
STRC in different sources of human hematopoietic cells.

Human cells regenerated**

| Cells | Total after 3 weeks | 6-8 weeks |
|---|---|---|
| transplanted* | (% GlycA⁺) | (% CD34⁻CD19/20⁺) |
| mPB | 1.0 ± 0.6 × 10⁶ (100) | 3.8 ± 2.4 × 10⁶ (87) |
| BM | 1.2 ± 0.3 × 10⁶ (62) | 7.6 ± 2.1 × 10⁶ (62) |
| GB | 7.0 ± 2.1 × 10⁶ (69) | 23.8 ± 7.7 × 10⁶ (79) |

*CD34⁺ cell-enriched samples from the different sources were transplanted into NOD/SCID-β₂M⁺ mice.
**Numbers of total human CD45/71⁺ cells in the murine BM per 10⁵ CD34⁺ cells injected are shown.
Values represent mean ± SEM from 3 mPB, 2 BM and 4 CB experiments.

### FULL CITATIONS FOR REFERENCES REFERRED TO IN THE SPECIFICATION

1. Glucknan E, Broxmeyer H E, Auerbach A D, et al. Hematopoietic reconstitution in a patient with fanconi's anemia by means of umbilical-cord blood from an HLA-identical sibling. N Engl J Med. 1989;321:1174-1178.

2. Pflumio F, Izac B, Katz A, Shultz L D, Vainchenker W, Coulombel L. Phenotype and function of human hematopoietic cells engrafting immune-deficient CB17-severe combined immunodeficiency mice and nonobese diabetic-severe combined immunodeficiency mice after transplantation of human cord blood mononuclear cells. Blood. 1996;88:3731-3740.

3. Conneally E, Cashman J, Petzer A, Eaves C. Expansion in vitro of transplantable human cord blood stem cells demonstrated using a quantitative assay of their lympho-myeloid repopulating activity in nonobese diabetic-scid/scid mice. Proc Natl Acad Sci USA. 1997;94:9836-9841.

4. Wang J C Y, Doedens M, Dick J E. Primitive human hematopoietic cells are enriched in cord blood compared with adult bone marrow or mobilized peripheral blood as measured by the quantitative in vivo SCID-repopulating cell assay. Blood. 1997;89:3919-3924.

5. van Hennik P B, de Koning A E, Ploemacher R E. Seeding efficiency of primitive human hematopoietic cells in nonobese diabetic/severe combined immune deficiency mice: implications for stem cell frequency assessment. Blood. 1999;94:3055-3061.

6. Cashman J D, Eaves C J. High marrow seeding efficiency of human lympho-myeloid repopulating cells in irradiated NOD/SCID mice. Blood.(in press).

7. To L B, Haylock D N, Simmons P J, Juttner C A. The biology and clinical uses of blood stem cells. Blood. 1997;89:2233-2258.

8. Wu A M, Till J E, Siminovitch L, McCulloch E A. Cytological evidence for a relationship between normal hematopoietic colony-forming cells and cells of the lymphoid system. J Exp Med. 1968;127:455-464.

9. Jordan C T, Lemischka I R. Clonal and systemic analysis of long-term hematopoiesis in the mouse. Genes Dev. 1990;4:220-232.

10. Keller G, Snodgrass R. Life span of multipotential hematopoietic stem cells in vivo. J Exp Med. 1990;171:1407-1418.

11. Morrison S J, Weissman I L. The long-term repopulating subset of hematopoietic stem cells is deterministic and isolatable by phenotype. Immunity. 1994;1:661-673.

12. Kondo M, Weissman I L, Akashi K. Identification of clonogenic common lymphoid progenitors in mouse bone marrow. Cell. 1997;91:661-672.

13. Akashi K, Traver D, Miyamoto T, Weissman I L. A clonogenic common myeloid progenitor that gives rise to all myeloid lineages. Nature. 2000;404:193-197.

14. Eaves C J, Eaves A C. Anatomy and physiology of hematopoiesis. In: Pui C-H, ed. Childhood Leukemias. NY: Cambridge University Press; 1999:53-71.

15. Civin C I, Almeida-Porada G, Lee M-J, Olweus J, Terstappen L W M M, Zanjani E D. Sustained, retransplantable, multilineage engraftment of highly purified adult human bone marrow stem cells in vivo. Blood. 1996;88:4102-4109.

16. Cashman J D, Lapidot T, Wang J C Y, et al. Kinetic evidence of the regeneration of multilineage hematopoiesis from primitive cells in normal human bone marrow transplanted into immunodeficient mice. Blood. 1997;89:4307-4316.

17. Cashman J, Bockhold K, Hogge D E, Eaves A C, Eaves C J. Sustained proliferation, multi-lineage differentiation and maintenance of primitive human haematopoietic cells in NOD/SCID mice transplanted with human cord blood. Br J Haematol. 1997;98:1026-1036.

18. Larochelle A, Vormoor J, Hanenberg H, et al. Identification of primitive human hematopoietic cells capable of repopulating NOD/SCID mouse bone marrow: implications for gene therapy. Nat Med. 1996;2:1329-1337.

19. Bhatia M, Wang J C Y, Kapp U, Bonnet D, Dick J E. Purification of primitive human hematopoietic cells capable of repopulating immune-deficient mice. Proc Natl Acad Sci USA. 1997;94:5320-5325.

20. Bonnet D, Bhatia M, Wang J C Y, Kapp U, Dick J E. Cytokine treatment of accessory cells are required to initiate engraftment of purified primitive human hematopoietic cells transplanted at limiting doses into NOD/SCID mice. Bone Marrow Transplant. 1999;23:203-209.

21. Bhatia M, Bonnet D, Murdoch B, Gan O I, Dick J. A newly discovered class of human hematopoietic cells with SCID-repopulating activity. Nat Med. 1998;4:1038-1045.

22. Nakamura Y, Ando K, Chargui J, et al. Ex vivo generation of CD34⁺ cells from CD34⁻ hematopoietic cells. Blood. 1999;94:4053-4059.

23. Christianson S W, Greiner D L, Hesselton R, et al. Enhanced human CD4⁺ T cell engraftment in β₂-microglobulin-deficient NOD-scid mice. J Immunol. 1997;158:3578-3586.

24. Kollet O, Peled A, Byk T, et al. β₂ microglobulin-deficient (B2m^{null}) NOD/SCID mice are excellent recipients for studying human stem cell function. Blood. 2000;95:3102. Published.

25. Holyoake T L, Nicolini F E, Eaves C J. Functional differences between transplantable human hematopoietic stem cells from fetal liver, cord blood, and adult marrow. Exp Hematol. 1999;27:1418-1427.

26. Monette F C, DeMello J B. The relationship between stem cell seeding efficiency and position in cell cycle. Cell Tissue Kinet. 1979;12:161-175.

27. Szilvassy S J, Meyerrose T E, Grimes B. Effects of cell cycle activation on the short-term engraftment properties of ex vivo expanded murine hematopoietic cells. Blood. 2000;95:2829-2837.

28. Habibian H K, Peters S O, Hsieh C C, et al. The fluctuating phenotype of the lymphohematopoietic stem cell with cell cycle transit. J Exp Med. 1998;188:393-398.

29. Glimm H, Oh I, Eaves C. Human hematopoietic stem cells stimulated to proliferate in vitro lose engraftment potential during their S/G₂/M transit and do not re-enter G₀. Blood. 2000;96(13):4185-4193.

30. Van der Loo J C M, Hanenberg H, Cooper R J, Luo F-Y, Lazaridis E N, Williams D A. Nonobese diabetic/severe combined immunodeficiency (NOD/SCID) mouse as a model system to study the engraftment and mobilization of human peripheral blood stem cells. Blood. 1998;92:2556-2570.

31. Magli M C, Iscove N N, Odartchenko N. Transient nature of early haematopoietic spleen colonies. Nature. 1982;295:527-529.

32. Ploemacher R E, Brons N H C. Isolation of hemopoietic stem cell subsets from murine bone marrow: I. Radioprotective ability of purified cell suspensions differing in the proportion of day-7 and day-12 CFU-S. Exp Hematol. 1988;16:21-26.

33. Gothot A, Van der Loo J C M, Clapp W, Srour E F. Cell cycle-related changes in repopulating capacity of human mobilized peripheral blood CD34⁺ cells in non-obese diabetic/severe combined immune-deficient mice. Blood. 1998;92:2641-2649.

34. Kim H J, Tisdale J F, Wu T, et al. Many multipotential gene-marked progenitor or stem celll clones contribute to hematopoiesis in nonhuman primates. Blood. 2000;96:1-8.

35. Uchida N, Aguila H L, Fleming W H, Jerabek L, Weissman I L. Rapid and sustained hematopoietic recovery in lethally irradiated mice transplanted with purified Thy-1.1^{lo} Lin⁻ Sca-1⁺ hematopoietic stem cells. Blood. 1994;83:3758-3779.

36. Zanjani E D, Ahmeida-Porada G, Livingston A G, Flake A W, Ogawa M. Human bone marrow CD34⁻ cells engraft in vivo and undergo multilineage expression that includes giving rise to CD34⁺ cells. Exp Hematol. 1998;26:353-360.

We claim:

1. A method of detecting a short term repopulating human hematopoietic cell that can produce myeloid cells in NOD/SCID-β₂M^{-/-} mice comprising (a) transplanting human hematopoietic cells in a NOD/SCID-β₂M^{-/-} mouse and (b) detecting human erythroid cells at approximately three weeks post-transplant.

2. A method according to claim 1 wherein the short term repopulating cells are CD34⁺CD38⁺.

3. A method according to claim 1 or 2 wherein the human erythroid cells are detected in a sample of bone marrow from the mouse.

4. A method of detecting a short term repopulating human hematopoietic cell that can produce myeloid and lymphoid cells in NOD/SCID-β₂M^{-/-} mice comprising (a) transplanting human hematopoietic cells in a NOD/SCID-β₂M^{-/-} mouse and (b) detecting human myeloid and lymphoid cells at approximately six to eight weeks post-transplant.

5. A method according to claim 4 wherein the short term repopulating cells retain their engraftment potential when they proliferate.

6. A method according to claim 4 to 5 wherein the human myeloid and lymphoid cells are detected in a sample of bone marrow from the mouse.

7. A method according to any one of claims 1 to 6 wherein the transplanted human hematopoietic cells are from peripheral blood, bone marrow or cord blood.

8. A method according to any one of claims 1 to 3 wherein the erythroid cells are detected using Fluorescence Activated Cell Sorting (FACS).

9. A method according to any one of claims 4 to 6 wherein the myeloid or lymphoid cells are detected using Fluorescence Activated Cell Sorting (FACS).

10. A method of assessing the short term repopulating potential of human hematopoietic cells comprising:
(a) administering the human hematopoietic cells to a NOD/SCID-β₂M^{-/-} mouse;
(b) obtaining a sample from the mouse at approximately 3 weeks after step (a);
(c) assaying the sample for human short term repopulating cells-myeloid (STRC-M) wherein the presence of STRC-M indicates that the human hematopoietic cells have short term repopulating potential.

11. A method according to claim 10 wherein the STRC-M are assayed by detecting human erythroid cells in the sample.

12. A method of assessing the short term repopulating potential of human hematopoietic cells comprising:
(a) administering the human hematopoietic cells to a NOD/SCID-β₂M^{-/-} mouse;
(b) obtaining a sample from the mouse at approximately 6-8 weeks after step (a);
(c) assaying the sample for human short term repopulating cells-lympho-myeloid (STRC-ML) wherein the presence of STRC-ML indicates that the human hematopoietic cells have short term repopulating potential.

13. A method according to claim 12 wherein the STRC-ML are assayed by detecting human myeloid and lymphoid cells in the sample.

14. A method of assessing the toxicity of a drug on human hematopoietic cells comprising:
(a) exposing the human hematopoietic cells to the drug;
(b) administering the cells from (a) to a NOD/SCID-β₂M^{-/-} mouse;
(c) obtaining a sample from the mouse at approximately 3 weeks after step (b);
(d) assaying the sample for human short term repopulating cells-myeloid (STRC-M) wherein the presence of STRC-M at levels approximately equal to that of untreated cells indicates that the drug is not toxic to these cells.

15. A method according to claim 14 wherein the STRC-M are assayed by detecting human erythroid cells in the sample.

16. A method of assessing the toxicity of a drug on human hematopoietic cells comprising:
(a) exposing the human hematopoietic cells to the drug;
(b) administering the cells from (a) to a NOD/SCID-β₂M^{-/-} mouse;
(c) obtaining a sample from the mouse at approximately 6 to 8 weeks after step (b);
(d) assaying the sample for human short term repopulating cells-lympho-myeloid (STRC-ML) wherein the presence of STRC-ML at levels approximately equal to that of untreated cells indicates that the drug is not toxic to these cells.

17. A method according to claim 16 wherein the STRC-ML are assayed by detecting human myeloid and lymphoid cells in the sample.

18. A method of assessing the viability of a human hematopoietic cell sample comprising:
(a) administering the human hematopoietic cells to a NOD/SCID-β₂M^{-/-} mouse;
(b) obtaining a sample from the mouse at approximately 3 weeks after step (a);
(c) assaying the sample for human short term repopulating cells-myeloid (STRC-M) wherein the presence of STRC-M indicates that the sample has viable short term repopulating cells.

19. A method according to claim 18 wherein the STRC-M are assayed by detecting human erythroid cells in the sample.

20. A method of assessing the short term repopulating potential of human hematopoietic cells comprising:
(a) administering the human hematopoietic cells to a NOD/SCID-β₂M^{-/-} mouse;
(b) obtaining a sample from the mouse at approximately 6 to 8 weeks after step (a);
(c) assaying the sample for human short term repopulating cells-lympho-myeloid (STRC-ML) wherein the presence of STRC-ML indicates that the sample has viable short term repopulating cells.

21. A method according to claim 20 wherein the STRC-ML are assayed by detecting human myeloid and lymphoid cells in the sample.

22. A method according to any one of claims 1-3, 7, 8, 11, 15 or 19 wherein the human erythroid cells are detected by detecting glycophorin A positive or CD71 positive cells in the sample.

23. A method according to any one of claims 4-7, 9, 13, 17 or 21 wherein the myeloid and lymphoid cells are detected by detecting CD34⁻CD19/20⁺ cells and glycophorin A⁺ or CD41⁺ or CD15/66b⁺ cells in the sample.

24. A short term repopulating human cell that can produce myeloid cells in NOD/SCID-β₂M^{-/-} mice.

25. A short term repopulating human cell according to claim 24 characterized by the rapid production of human erythroid cells at approximately three weeks post-transplant of human hematopoietic cells in a NOD/SCID-β₂M^{-/-} mouse.

26. A short term repopulating human cell according to claim 24 or 25 wherein the cells are CD34⁺CD38⁺.

27. A short term a short term repopulating human cell that can produce myeloid and lymphoid cells in NOD/SCID-β₂M^{-/-} mice.

28. A short term repopulating cell according to claim 27 is characterized by a transient burst in human lymphoid and myeloid cell production that peaks at six to eight weeks post transplant of human hematopoietic cells in a NOD/SCID-β₂M^{-/-} mouse.

29. A short term repopulating cell according to claim 27 or 28 wherein the cells retain their engraftment potential when they proliferate.
